(19) United States
(12) Patent Application Publication
(10) Pub. No.: US 2011/0071435 A1
(43) Pub. Date: Mar. 24, 2011

(54) Method and System for Coupling an Extension Wire to a Guidewire

(75) Inventors: Noam SHAMAY, Rehovot (IL); German Aleynikov, Ashdod (IL); Ofer Zigman, Rehovot (IL)

(73) Assignee: OVALUM Ltd., Rehovot (IL)

(21) Appl. No.: 12/871,863

(22) Filed: Aug. 30, 2010

Related U.S. Application Data
(63) Continuation of application No. PCT/IL2009/000219, filed on Feb. 26, 2009.

(30) Foreign Application Priority Data
Feb. 28, 2008 (GB) 0803656.8

(51) Int. Cl. A61M 25/09

(52) U.S. Cl. 600/585

(57) ABSTRACT

A method and a system using a helicoidal structure wound out of a plurality of metallic wires into a universal connector for reversibly coupling a distal end portion of an extension wire to a proximal end portion of a guide wire. The universal connector is configured for receiving the proximal end portion of the guide wire into the connector lumen and is made as a single-piece unit built to sustain increased separation forces tending to disconnect the extension wire from the guide wire. The universal connector accommodates coupling to different configurations of commercially available guide wire proximal terminations.

### RELATED APPLICATIONS

This application is a Continuation Application of International Application No. PCT/12009/000219 filed on 26 Aug. 2009. This application claims priority from Great Britain application no. 0803656.8 filed Feb. 28, 2008, the entire content of which is hereby incorporated by reference.

### FIELD OF THE INVENTION

The embodiments of the present invention relate to connection means for coupling an extension wire to a guide wire, and in particular, to a method and to a system wherein a connector is configured as a single-piece unit built to sustain increased separation forces tending to disconnect the extension wire from the guide wire.

### BACKGROUND OF THE INVENTION

Connection means for coupling an extension wire to a guide wire are well known per se. Such connection means usually include a spring coil wound from a single spring wire, operating in association with some kind of tube, as recited in the following patents and patent applications.

European Patent Application No. EP 0 799 625 to Lorenzo, recites a coiled spring connecting a guide wire and an extension wire, and a tube disposed over the coiled spring. The tube is attached to slide over the extension wire and to engage the tube. The tube may slide to expand the coil and release the wire.

European Patent Application No. EP 0 799 624 to Sasamine et al., recites a guide wire, an extension wire to which a hypotube is permanently affixed, and a spring coil. The hypotube has a tongue to which the spring coil is permanently affixed. The spring coil is configured to receive the extension wire in frictional fit.

U.S. Pat. No. 5,421,348 to Lanard recites a guide wire extension system including a guide wire, an extension guide wire, and a connecting assembly mounted on the extension guide wire. The connecting assembly includes a coiled spring arranged to receive and lock with the initial guide wire and a hypotube received loosely in the hypotube. The coiled spring has one end fixed to the tube and another free end. The tube is rotated to withdraw the coiled spring and disconnect the extension wire from the guide wire.

U.S. Pat. No. 5,197,486 to Frassica et al., recites a guide wire extension system with a guide wire, an extension guide wire, and a connecting assembly including a coiled spring, a small diameter tube and a rotatable connection disposed between the guide wire and the tube.

U.S. Pat. No. 5,113,872 to Jahrmarkt et al., recites a guide wire extension system with an extension guide wire, a guide wire, a connecting assembly including a coiled spring, and a small diameter tube associated with the coiled spring.

Further examples include: U.S. Pat. No. 5,415,178 to His et al., U.S. Pat. No. 5,701,911 to Sasamine et al., U.S. Pat. No. 5,788,653 to Lorenzo, and U.S. Pat. No. 5,117,838 to Palmer, et al., all have in common a coil spring wound from a single spring wire and a tube of some kind.

However, a single spring wire is able to sustain but limited forces applied thereon. None of the disclosures of the background art ensures a superior separation force, to prevent the connection means to release grip on the guide wire in response to a pull as low as about one Newton, or 1 N. This means that the application of a relatively low pull-apart force disconnects the guide wire from the extension wire. Increased resistance to superior separation forces is not obtainable with a coil spring wound out of a single spring wire.

It would be advantageous to provide an extension wire having a connection means that is not restricted to low separation forces, but is configured to offer a pull-apart separation force exceeding the low retention force of commercially available systems and preferably having a pull-apart separation force much higher than required by International Standards for connection means of larger diameter.

In addition, it would be advantageous to provide an extension wire having connection means configured as a naked unsupported wound structure of wires without further implement(s), such as support tubes, for coupling to a guide wire.

Moreover, it would be advantageous to provide an extension wire able to be coupled by use of a universal connector to any existing configuration of guide wire proximal termination, or at least to the majority of commercially available guide wires.

One problem with existing miniature coil spring connectors wound from a single spring wire is the relatively low pull-apart force at which the connection fails, thereby causing release and separation to occur between the guide wire and the extension wire. When a pulling force is exerted on a miniature coil spring, at first, and even under even a relatively low-force pull, the coils expand beyond the elastic limit and suffer a permanent-set plastic deformation. Actually, a low-force pull suffices to first plastically stretch the loops of thin wire from which the helical coil spring connector is made, and second to cause release of grip, whereby the connection fails and the guide wire disconnects from the extension wire.

Comparison tests were conducted between a commercially available type of connector and the universal connector of the present claimed invention, details about which are listed in Table 1 hereinbelow.

A first set of tensile strength tests was conducted on five samples of a same sturdy commercially available coil spring connector wound from a single 304 stainless steel wire, with the connector coupling a short portion of guide wire to a short portion of extension wire. The single-wire coil connectors had an outer diameter of 0.0134″+/−0.0005″, or about 0.340 mm+/−0.000127 mm, a pitch of 0.1 mm, and a single spring wire of rectangular cross section of 0.1×0.05 mm. The test was performed on a Check Line mechanical test stand made by the Electromatic Equipment Co. Inc. of Cedarhurst, N.Y., USA, model FGS50PUH, operating at the rate of 40 mm/min, until failure by release of the connection. Inches are specified as ″, millimeters are indicated as mm, and a Newton of force is designated as N.

Results for a single-wire coiled spring showed that the highest disconnection force reached was 1.992 N, with the lowest release or coupling failure force occurring at 1.049 N. The average failure force was 1.483 N+/−0.4364 N, or roughly, 1.5 N. Forces were measured with a force gage model FG5000A, made by the Lutron Electronic Entreprise Co. Ltd. of Taipei, Taiwan.

It turned out that a single spring wire of such small cross-section is too weak to support high pull stresses applied thereon, and thus prohibits the implementation of a reliable helical compression coil spring connector.

### SUMMARY OF THE INVENTION

A solution for enhancing the release force under traction may be provided by use of a helicoidal wire-tube wound from a plurality of metallic spring wires, thus having the general configuration of a helical structure wound from more than one single wire. Such a helicoidal structure may include various configurations all having multiple spring wires.

The solution is provided by a universal connector configured as a sleeve having multiple spring wires, or as a hollow stranded wire tube twisted out of a plurality of metallic wires tightly stranded with a high pitch and in gapless contact with each other. Such a universal connector is also referred to hereinbelow as a helical hollow wire-body, or sleeve-body of wires, as best seen in FIG. 2.

A second set of tensile strength tests was conducted on five sleeve-bodies made from 304 stainless steel wire coupling a short portion of guide wire to a short portion of extension wire. The tested sleeve-bodies of wires, as supplied by the Asahi Intec Co., Ltd. of Nagoya in Japan, had an outer diameter of 0.0136″+/−0.0003″, or about 0.350 mm+/−0.00762 mm, and 12 left-hand wound wires with a pitch of 1.04 mm+/−0.1 mm, and a spring wire diameter of 0.002″ or about 0.05 mm. The second test was performed on the same motorized tensile machine at the same rate of 40 mm/min, until release failure of the connection.

Results showed that the highest disconnection force reached for a multi-wire sleeve-body was 13.259 N, with the lowest coupling failure force being 7.031 N. The average failure force was 9.632 N+/−3.114 N, or about 9.6 N.

Comparison hence shows that a high-pitched sleeve-body of wires may sustain separation forces superior by about a six-fold over the separation forces that a single-wire coil spring is able to support.

TABLE 1

| | Connector | Wire cross- | Pitch | No. of | Average |
|---|---|---|---|---|---|
| | O.D. | section | distance | wires | release |
| | [mm] | [mm] | [mm] | [#] | [N] |
| Single-wire | 0.350 | 0.1 × 0.05 | 0.1 | 1 | ~1.5 |
| 304 S.S. | | | | | |
| spring | | | | | |
| Tolerance +/− | 0.000127 | 0.002 | 0.001 | | |
| 304 S.S. | 0.350 | 0.050 | 1.04 | 12 | ~9.6 |
| multi-wire | | | | | |
| connector | | | | | |
| Tolerance +/− | 0.00762 | 0.00762 | 0.1 | | |

It is an object of the embodiments described hereinbelow to provide a system and a method for releasably coupling an extension wire 200 to a guide wire 300 by use of a universal connector 100. The guide wire 300 comprises a guide wire exterior diameter GWD, a guide wire body 302 having a guide wire distal portion 304, and a guide wire proximal portion 306 including a guide wire proximal extremity 308 having reduced dimensions relative to the guide wire exterior diameter and a proximal tip section 310. The extension wire 200 comprises an extension wire exterior diameter XWD, an extension wire body 202 including an extension wire proximal portion 204 and an extension wire distal portion 206 which terminates in an extension wire distal extremity 208.

The universal connector 100 comprises a connector body 102 of hollow helicoidal structure including a connector length L having a connector exterior diameter UCD, a connector distal portion 104 having a connector distal opening 108 and a connector proximal portion 106 having a connector proximal opening 110, and a connector lumen 112 extending throughout the length of the universal connector. The connector distal opening 108 is configured for receiving the guide wire proximal extremity 308 into the connector lumen 112 for releasable coupling to the guide wire proximal portion 306, and the connector proximal portion 106 is fixedly coupled to the extension wire distal portion 206.

The method is characterized by comprising the steps of configuring the universal connector 100 out of a length of a plurality of metallic wires 114 stranded together with a high pitch distance 116 and twisted for tight gapless contact with each other. The connector distal portion 104 is configured as an unconfined cantilevered self-supporting unit wherein the plurality of metallic wires 114 operates in mutual association. Thereby, when the guide wire 300 is coupled to the universal connector 100, a superior pull-apart resistance force is provided by the plurality of metallic wires against separation forces operating to extract the guide wire 300 out of the universal connector 100.

It is also an object of the embodiments described hereinbelow to provide a universal connector wherein the plurality of metallic wires provide superior pull-apart resistance force relative to the pull-apart resistance force provided by a connector configured to include a single wire helical coil spring.

It is another object of the embodiments described hereinbelow to provide a universal connector wherein the high-pitch distance is equal to at least the exterior diameter of the universal connector. Moreover, the high-pitch distance is constant or varies over the length of the universal connector.

It is still another object of the embodiments described hereinbelow to provide a universal connector wherein the exterior diameter of the universal connector is selected from a group including of a diameter smaller, equal or larger than the exterior diameter of the guide wire.

It is a further object of the embodiments described hereinbelow to provide a universal connector wherein each metallic wire 114 out of the plurality of metallic wires 114 has a wire cross-section shape 118 selected from a group including of circular, square, rectangular, trapezoidal, and oval cross-section shapes. Furthermore, each metallic wire 114 out of the plurality of metallic wires 114 may have a same wire cross-section shape 118 or a different wire cross-section shape and may be made from the same material or from a different material.

It is yet another object of the embodiments described hereinbelow to provide a universal connector wherein the proximal connector portion is fixedly coupled to the distal portion of the extension wire.

It is another object of the embodiments described hereinbelow to provide a universal connector wherein the proximal connector portion is fixedly coupled to the distal portion of the extension wire. In addition, the universal connector is configured as a hollow helical wire-body sleeve.

It is yet another object of the embodiments described hereinbelow to provide a universal connector wherein the connector distal opening 108 and the connector lumen 112 accommodate reception therein of guide wire proximal portions 306 having different proximal tip section 310 configurations, for secure releasable retention.

It is still another object of the embodiments described hereinbelow to provide a universal connector wherein a sleeve 122 of flexible film of material covers at least the length of the universal connector for enhancing smooth passage thereover of implements. Preferably, the length L of the connector body 102 is coated with a coat 122 of lubricant for enhancing smooth passage thereover of implements.

It is still one more object of the embodiments described hereinbelow to provide a universal connector wherein the flexibility of the universal connector 100 is controlled.

### BRIEF DESCRIPTION OF THE DRAWINGS

Non-limiting embodiments of the invention will be described with reference to the following description of exemplary embodiments, in conjunction with the figures. The figures are generally not shown to scale and any measurements are only meant to be exemplary and not necessarily limiting. In the figures, identical structures, elements, or parts that appear in more than one figure are preferably labeled with a same or similar number in all the figures in which they appear, in which:

FIG. 1 is a schematic illustration depicting a universal connector disposed on an extension wire in the presence of a guide wire,

FIG. 2 shows the universal connector in more detail,

FIG. 3 depicts details of an extension wire distal portion, the universal connector, and a guide wire proximal portion,

FIG. 4 illustrates another embodiment of an extension wire distal portion in direct coupling with a universal connector, and

FIG. 5 presents an example of a connector sleeve, or a connector coating.

### DETAILED DESCRIPTION OF THE DRAWINGS

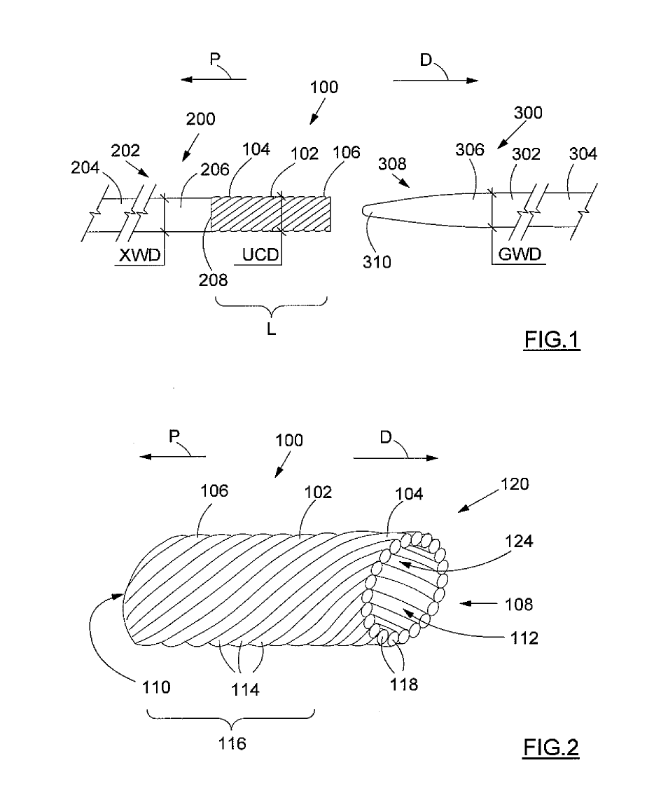

FIG. 1 shows an exemplary embodiment of a universal connector 100, and FIG. 2 shows the universal connector only.

The universal connector 100 may be described generally as a flexible hollow helicoidal structure, or as a hollow helical wire-body sleeve, which is coupled to an extension wire 200, appropriately configured for releasably coupling the extension wire 200 to a guide wire 300. In FIG. 1, the guide wire 300 is disposed distally relative to the universal connector 100, which in turn, is disposed distally relative to the extension wire 200.

Distal or distal direction and proximal or proximal direction are indicated in the FIGS. by arrows marked as, respectively, D and P.

FIG. 2 illustrates the universal connector 100, which is of generally hollow helicoidal structure and may be described as a helical hollow wire-body, or as a wire-stranded hollow tube having a plurality of metallic wires that are wound tightly together.

The flexible universal connector 100 may include a connector body 102 having a connector distal portion 104, a connector proximal portion 106, a connector length L, and a connector exterior diameter UCD. The connector distal portion 104 has a connector distal opening 108, and the connector proximal portion 106 has a connector proximal opening 110. A connector lumen 112 extends throughout the length L of the universal connector 100, from the connector distal opening 108 to the connector proximal opening 110.

The flexible universal connector 100 may include a plurality of lengths of metallic wires 114 stranded together with a high pitch distance 116 and twisted in pre-stress for tight gapless contact with each other. The connector distal portion 104 is actually configured as an unconfined cantilevered self-supporting unit wherein the plurality of metallic wires 114 operates in mutual association.

A pitch distance of the universal connector 100 is considered as the distance from center to center of two adjacent metallic wires. For the universal connector 100, a high-pitch distance 116 may be regarded as a distance that is at least equal to the exterior diameter UCD of the universal connector 100. However, the high pitch distance may also be practical with a pitch that is preferably more than one exterior diameter UCD, such as three times, or five times the exterior diameter UCD for example. The high-pitch distance 116 may be constant or may vary over the length L of the universal connector 100.

Each wire 114 out of the plurality of metallic wires 114 has a wire cross-section shape 118. The shape of the wire cross-section shape 118 may be selected as any desired geometrical shape, preferably out of commercially available wires, but any cross-section shape that may be drawn is feasible. For example, a wire cross-section shape 118 may be selected to be of circular, square, rectangular, trapezoidal, oval, or of other cross-section shape. However, each metallic wire 114 out of the plurality of metallic wires 114 may be selected to have, a same wire cross-section shape 118, a different wire cross-section shape or a combination of -section shapes.

The plurality of metallic wires 114 at the connector distal opening, indicated as a connector free-end 120 in FIG. 2, are prevented from unwinding by being coupled together. Such coupling of the strands of the universal connector 100, or metallic wires 114, is preferably performed by laser welding, providing a thin layer of welded material to firmly hold the very extremity of the twisted metallic wires at the connector free-end 120 in tight gapless contact with each other.

The metallic wires 114 may be selected as wires made of the same material or made of different materials. For example, such materials may include austenitic stainless steels, shape-memory alloys, or super-elastic alloy stainless steel wires, such as a nitinol. Preferably, the metallic wires 114 are disposed as a single layer of wires along the circumference of the universal connector 100, leaving a central connector lumen 112, as shown in FIG. 2. The number of metallic wires 114 may range from six to 20 for example, preferably 18, and more preferably 12, depending on the universal connector exterior diameter UCD. Moreover, the direction of winding of the plurality of metallic wires 114 may be chosen to be clockwise or anti-clockwise.

In FIG. 1, the extension wire 200 is depicted to include an extension wire exterior diameter XWD, an extension wire body 202 having an extension wire proximal portion 204 and an extension wire distal portion 206 which terminates in an extension wire distal extremity 208, to which the universal connector 100 is fixedly coupled.

The extension wire proximal portion 204 may be coupled to the extension wire distal portion 206 by any means known in the art, such as for example butt-coupling, stem-coupling, stem-and-support coupling, alone or in any practical combination thereof. Furthermore, secure fixed fastening of the connector 100 to the extension wire 200 may be provided by means known in the art, such as for example welding, brazing, soldering, gluing, or shrink fit.

Still in FIG. 1, the guide wire 300 is shown to include a guide wire exterior diameter GWD, a guide wire body 302 having a guide wire distal portion 304 and a guide wire proximal portion 306, which terminates in a guide wire proximal extremity 308 having reduced dimensions relative to the guide wire exterior diameter, and terminating in a guide wire proximal tip section 310. This means that the guide wire proximal extremity 308 has a reduced exterior diameter relative to the guide wire body 302, which exterior diameter diminishes gradually towards the proximal tip section 310.

Once the guide wire 300 is coupled to the universal connector 100, the multi-wire high-pitch helicoidal configuration of the universal connector will provide a superior pull-apart resistance force against separation forces operating to extract the guide wire 300 out of the universal connector 100. In other words, the plurality of metallic wires 114 provide superior pull-apart resistance force relative to the pull-apart resistance force provided by a background art connection means configured to include a helical spring coil wound with a single spring wire.

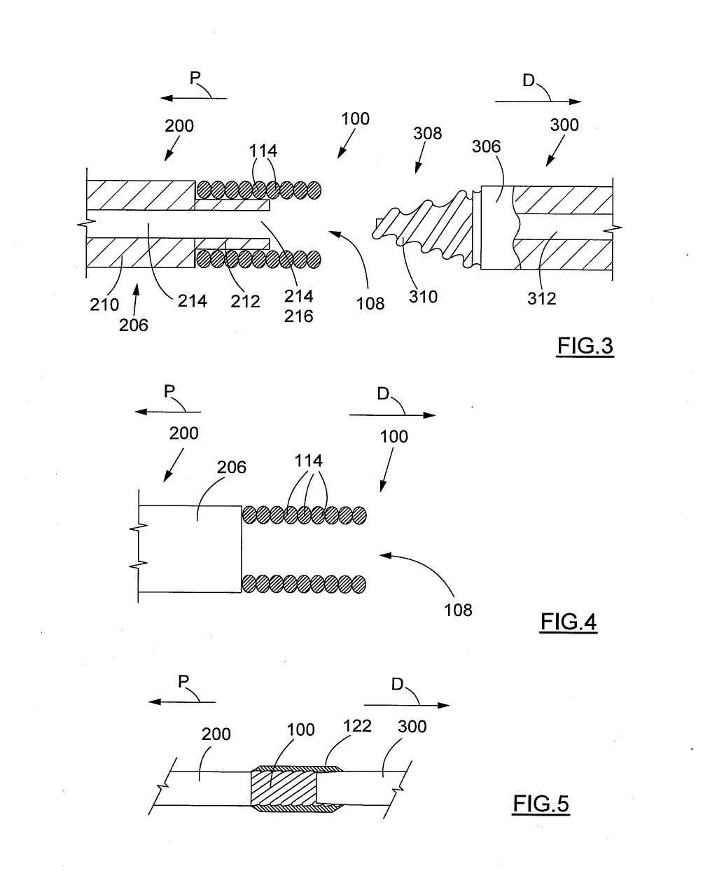

As already described hereinabove, the extension wire proximal portion 204 may be coupled to the extension wire distal portion 206 by any means known in the art, such as for example butt-coupling, stem-coupling, stem-and-support coupling respectively, or by any practical combination thereof. FIG. 3 depicts an example of a possible coupling embodiment to fixedly secure the flexible universal connector 100 to the extension wire 200.

In FIG. 3, the extension wire distal end portion may be provided with a solid or with a hollow extension wire distal appendage 210, or with a hollow tube ending distally in a coaxial extension wire distal support 212. The extension wire distal support 212 is inserted into the connector proximal opening 110 in fit with the connector lumen 112, and forms a shoulder with the extension wire distal appendage onto which the connector proximal portion 106 abuts, to enhance coupling of the universal connector 100 to the extension wire 200. The extension wire distal support 212 may be cylindrical or have any other desired shape suitable to facilitate any process, such as manufacture, assembly, and use.

The extension wire distal support 212 is preferably an integral portion of the extension wire distal appendage 210, or is a separate portion attached thereto to provide a support for the universal connector 100. Alternatively, the extension wire distal appendage 210 is either a separate portion fixedly coupled to the extension wire 200, or an integral portion of the extension wire 200.

When supported by an appropriately configured extension wire distal support 212, or when coupled in butt-retention to the extension wire distal extremity 208, the universal connector 100 may have a universal connector exterior diameter UCD that is smaller, equal, or larger than the guide wire exterior diameter GWD or the extension wire exterior diameter XWD. This means that the universal connector 100, the extension wire 200, and the guide wire 300 may all have the same exterior diameter.

If desired, the extension wire distal appendage 210 and/or the extension wire distal support 212 may have an extension wire lumen 214 or an extension wire distal support lumen 216, or both, which disposed in continuation and in communication with a lumen provided and disposed in the interior of the extension wire 200. Else, the lumen provided in the extension wire 200 may be disposed in direct communication with the connector lumen 112 or the extension wire distal support lumen 216, or both. Furthermore, the guide wire 300 may have a guide wire lumen 312 pierced throughout and extending out of guide wire proximal tip section 310 to establish communication in continuation with a lumen provided and disposed in the interior of the guide wire 300. This means that when the guide wire 300 has a guide wire lumen 312 extending distally away from a tip opening of the guide wire proximal tip section 310, and the extension wire 200 has an extension wire lumen 214, or an extension wire distal support lumen 216, or both, extending distally up to and through the guide wire distal portion 304, then the connector lumen 112 provides a conduit for bi-directional continuous unimpeded passage from the extension wire lumen to the guide wire lumen, and vice versa. In other words, bi-directional communication may be established between the extension wire 200 and the guide wire 300 via the connector 100.

Furthermore, secure fixed fastening of the connector 100 to the extension wire 200 may be provided by means known in the art, such as for example welding, brazing, soldering gluing or shrink fit.

The universal connector 100 is universal in the sense that secure and reliable coupling between a guide wire 300 and an extension wire 200 may be achieved irrespectively of the configuration and shape of the guide wire proximal tip section 310 inserted into the connector lumen 112. For example, the universal connector 100 will accommodate various types of guide wire proximal tip sections 310, for example screw thread-ended, as shown in FIG. 3, or nipple-ended, as depicted in FIG. 1. Hence, the connector distal opening 108 and the connector lumen 112 accommodate reception therein of guide wire proximal portions 306 having different proximal tip section 310 configurations, for secure releasable retention.

In general, the guide wire proximal end portion may have a guide wire proximal extremity 308 featuring a reduction in exterior diameter relative to the guide wire exterior diameter GWD or to the connector lumen 112, which terminates in a guide wire proximal tip section 310. The reduction in exterior diameter may be gradual, decreasing monotonously, for example, from a guide wire nominal exterior diameter GWD of 0.355 mm, or 0.014 inches, down to 0.152 mm, or 0.006 inches. Sometimes, the guide wire proximal tip section 310 is terminated by a screw thread, or ends in any other type of appendage having a reduced diameter relative to the guide wire exterior diameter GWD or to the connecter lumen 112.

The flexibility of the universal connector 100 may be controlled to be stiffer or yielding, thus less rigid, according to desire. Flexibility is dependent on the material of the metallic wires 114, on the shape of their cross-section 118, on the pitch distance 116, on twisting and pre-stressing in tight gapless contact of the metallic wires 114, or on a mix of all of the above, for example, when the extension wire distal extremity 208 and the guide wire proximal tip section 310 are kept supported and separated apart by a portion of the connector body 102. Hence, the flexibility of the universal connector 100 may be controlled.

However, it is possible to configure the guide wire proximal tip section 310 to be thin enough to penetrate throughout the connector lumen 112, exit via the connector proximal opening 110, and penetrate into the extension wire lumen 214. When the guide wire proximal tip section 310 is seated into the extension wire lumen 214, it is the rigidity of the guide wire proximal portion 306 that is added to the rigidity of the universal connector 100, which thereby provides a stiffer universal connector.

If desired, only the length L of the universal connector 100, or also a portion of either one of both or of both the extension wire distal portion 206 and the guide wire proximal portion 306 may be covered with a thin connector sleeve 122 made from synthetic resin for example.

FIG. 5 illustrates a connector sleeve 122, which is shown out of scale in grossly exaggerated dimensions. A connector sleeve 122 may be implemented as a thin and flexible film of material, such as a polymer for example, that covers at least the universal connector for enhancing smooth transition passage of over-the-wire implements over and around the universal connector 100. It is noted that the length L of the connector body 102 may also be coated with a friction reducing coat of solid lubricant, such as Teflon, which is a registered Trademark, for enhancing smooth transition passage thereover of over-the-wire implements. The connector sleeve 122 may add but a mere 0.0001″, or 0.0254 mm to the universal connector exterior diameter UCD.

For operation, thus for releasably coupling of the guide wire 300 to the extension wire 200, the latter is gripped proximally and held in place relative to the guide wire 300. It suffices to manually introduce and translate the guide wire proximal portion 306 into the open connector distal opening 108, until firmly seated and retained therein.

In other words, when the guide wire proximal portion 306 is introduced by proximal translation into the universal connector 100, mutual contact is made in the connector lumen interior 124 with the metallic wires 114. Then, the guide wire proximal portion 306 is pushed proximally further into the connector lumen 112, and friction develops between the contacted metallic wires 114 and the contacted guide wire proximal portion 306. In response to the introduction therein of the guide wire proximal portion 306, the universal connector 100 may compress, whereby the connector lumen 112 may slightly expand and allow further proximal introduction therein of the guide wire proximal portion 306, until no further translation is possible.

Coupling of the extension wire 200 and of the guide wire 300 may also be carried out manually by mutual and relative translation and rotation. For example, the extension wire 200 may be translated and rotated toward and onto the guide wire 300, whereby the universal connector 100 will also rotate. Rotation may be carried out in the direction of uncoiling of the metallic wires 114 of the universal connector 100. Thereby, friction forces develop between the guide wire proximal portion 306 and the interior of the connector lumen 112, which friction forces will cause the universal connector 100 to slightly expand until translation therein cannot proceed further, to better grip the guide wire proximal portion 306.

At this stage the extension wire 200 is firmly coupled to the guide wire 300. Disconnection of the guide wire 300 from the universal connector 100 by the mere exertion of separation forces, or pull-apart forces operating to extract the guide wire 300 out of the universal connector, is not possible. This means that a superior pull-apart resistance force is provided by the plurality of metallic wires against separation forces operating to extract the guide wire 300 out of the universal connector 100. In other words, the plurality of metallic wires 114 provide superior pull-apart resistance force against separation forces relative to the pull-apart resistance force provided connection means having a single-wire helical coil spring.

When the guide wire 300 is pulled distally away for extraction out of the universal connector 100, which is still retained in place proximally, friction forces will urge the universal connector to slightly extend, but firm grip on the guide wire 300 will not be lost. The connector lumen interior 124 may slightly contract to further better grip and augment the forces firmly retaining the guide wire proximal portion 306 into the connector lumen interior.

To retrieve the guide wire 200 out of the universal connector 100, it is necessary to grip the universal connector at the connector distal opening 108, and then to compress the universal connector 100. Thereby, the interior of the connector lumen 112 may slightly expand, and allow the guide wire proximal portion 306 to be released and withdrawn in distal translation pull, away from the universal connector 100.

A deliberate manual commanded and controlled action is thus required to disconnect the guide wire proximal portion 306 from the universal connector 100, in contrast for example, with the recitation of Kontos et al., which requires a mere pull-apart, which pull-apart separation may happen either inadvertently or accidentally.

In comparison to background art connection means, as by Table 1 hereinabove, the helicoidal structure configured as a high-pitched sleeve-body of metallic wires 100, or universal connector 100, sustains separation forces superior by about a six-fold six over the separation forces that a single wire coil spring is able to sustain.

It is also possible to disengage the guide wire 300 from the universal connector 100 in another manner.

To disengage the guide wire proximal portion 306 out of the universal connector 100, it is sufficient to firmly manually hold the guide wire proximal portion in place, while rotating the extension wire 200 relatively thereto, in the direction of uncoiling of the universal connector and simultaneously extracting the universal connector proximally away from the guide wire 300.

When the extension wire distal portion 206 is rotated relative to the guide wire proximal portion 306 in opposite direction to the winding direction of the universal connector 100, the friction forces tend to unwind the metallic wires 114, whereby the connector lumen interior 124 may slightly expand, and thereby may allow withdrawal and disconnection by translation.

Once again, a deliberate manual commanded and controlled action is required to disconnect the guide wire proximal portion 306 from the universal connector 100, as opposed to a mere pull-apart traction force, which may happen either inadvertently or accidentally, such as with Kontos et al., for example.

In use, a practitioner may intuitively connect and disconnect the universal connector 100 from the guide wire 300 as by the description hereinabove.

The connector described hereinabove is thus a universal connector 100, which may be coupled to any existing guide wire 300 or at least to the majority of the existing guide wires, since the connector distal opening 108 is wide open and has a connector lumen 112 that is configured to receive therein the guide wire proximal portion 306. This means that at least a proximal portion of the guide wire proximal portion 306 must have external dimensions smaller at least by twice the thickness of the metallic wires 114 relative to the universal connector exterior diameter UCD. In simple words, at least the proximal portion of the guide wire proximal portion 306 must be appropriately dimensioned to penetrate into the connector distal opening 108.

### INDUSTRIAL APPLICABILITY

The method and the system described hereinabove are appropriate for implementation in industries manufacturing medical instrumentation.

It will be appreciated by persons skilled in the art that the present invention is not limited to what has been particularly shown and described hereinabove. For example, the functions of the extension wire distal portion 206 and of the guide wire proximal portion 306 may be interchanged, but the operation and use of the universal connector 100 remain the same. This means that the universal connector 100 may be fixedly coupled to the proximal portion 306, and that the distal portion 206 may be configured for releasable coupling to the universal connector 100.

Rather, the scope of the present invention is defined by the appended claims and includes both combinations and sub combinations of the various features described hereinabove as well as variations and modifications thereof which would occur to persons skilled in the art upon reading the foregoing description.

The invention is not limited by the embodiments described above which are presented as examples only but can be modified in various ways within the scope of protection defined by the appended patent claims.

What is claimed:

1. A method for releasably coupling an extension wire to a guide wire by use of a universal connector, the method comprising the steps of:
providing the guide wire with a guidewire proximal portion including a guide wire proximal extremity and a proximal tip section;
coupling a proximal portion of the connector in fixed connection to an extension wire distal portion, and opening a connector lumen extending from the connector proximal portion to a connector distal portion which ends in a connector distal opening for receiving therein the guide wire proximal portion;
cantilevering the connector distal portion as an unconfined self-supporting unit; and
introducing the guide wire proximal extremity into the lumen via the connector proximal opening for coupling the guidewire to the connector which has a length of a plurality of metallic wires stranded together with a high pitch distance and twisted for tight gapless contact with each other,
wherein, when the guidewire is coupled to the universal connector, a superior pull-apart resistance force is provided by the plurality of metallic wires against separation forces operating to extract the guidewire out of the universal connector.

2. The method according to claim 1, wherein when the guidewire is coupled to the universal connector, the plurality of metallic wires provide superior pull-apart resistance force relative to the pull-apart resistance force provided by a connector configured to include a single wire helical coil spring.

3. The method according to claim 1, wherein:
the guide wire has an exterior diameter,
the universal connector has a length, an exterior diameter, and a high-pitch distance that is:
equal to at least the exterior diameter of the universal connector, and
constant or varies over the length of the universal connector, and
the exterior diameter of the universal connector is selected from a group including a diameter smaller, equal or larger than the exterior diameter of the guidewire.

4. The method according to claim 1, wherein each metallic wire out of the plurality of metallic wires is selected as:
having a wire cross-section shape selected from a group including circular, square, rectangular, trapezoidal, and oval cross-section shapes,
having a same wire cross-section shape or a different wire cross-section shape, and
being made from the same material or from a different material.

5. The method according to claim 1, wherein the universal connector is configured as a hollow helical wire-body sleeve.

6. The method according to claim 1, wherein the connector distal opening and the connector lumen accommodate reception therein of guidewire proximal portions having different proximal tip section configurations, for secure releasable retention.

7. The method according to claim 1, wherein:
the universal connector has a length, and
a sleeve of flexible film of material covers at least the length of the universal connector for enhancing smooth passage thereover of implements.

8. The method according to claim 1, wherein:
the universal connector has a length and a connector body, and
the length of the connector body is coated with a coat of lubricant for enhancing smooth passage thereover of implements.

9. The method according to claim 1, wherein flexibility of the universal connector is controlled.

10. A guidewire extension system for releasably coupling a distal end portion of an extension wire to a guidewire by use of a universal connector,
wherein the guidewire comprises:
a guidewire exterior diameter, a guidewire body having a guidewire distal portion, and a guidewire proximal portion including a guidewire proximal extremity having reduced dimensions relative to the guidewire exterior diameter and a proximal tip section;
wherein the extension wire comprises:
an extension wire exterior diameter, an extension wire body including an extension wire proximal portion and an extension wire distal portion which terminates in an extension wire distal extremity; and
wherein the universal connector comprises:
a connector body of hollow helicoidal structure including a connector length having a connector exterior diameter, a connector distal portion having a connector distal opening and a connector proximal portion having a connector proximal opening, and a connector lumen extending throughout the length of the universal connector,
the connector distal opening being configured for receiving the guidewire proximal extremity into the connector lumen for releasable coupling to the guidewire proximal portion, and
the connector proximal portion being fixedly coupled to the extension wire distal portion;
wherein the universal connector is configured out of a length of a plurality of metallic wires stranded together with a high pitch distance and twisted for tight gapless contact with each other,
wherein the connector distal portion is configured as an unconfined cantilevered self-supporting unit and the plurality of metallic wires operate in mutual association, and
wherein, when the guidewire is coupled to the universal connector, a superior pull-apart resistance force is provided by the plurality of metallic wires against separation forces operating to extract the guidewire out of the universal connector.

11. The system according to claim 10, wherein the plurality of metallic wires provide superior pull-apart resistance force relative to the pull-apart resistance force provided by a connector configured to include a single wire helical coil spring.

12. The system according to claim 10,
wherein the high-pitch distance of the universal connector is equal to at least wherein the exterior diameter of the universal connector, and is constant or varies over the length of the universal connector, and
wherein the exterior diameter of the universal connector is selected from a group including a diameter smaller, equal or larger than the exterior diameter of the guidewire.

13. The system according to claim 10, wherein each metallic wire out of the plurality of metallic wires is selected as:
having a wire cross-section shape selected from a group including circular, square, rectangular, trapezoidal, and oval cross-section shapes,
having a same wire cross-section shape or a different wire cross-section shape, and
being made from the same material or from a different material.

14. The system according to claim 10, wherein the universal connector is implemented out of wire pertaining to the extension wire.

15. The system according to claim 10, wherein the universal connector is configured as a helical wire-stranded tube made out of a plurality of metallic wires.

16. The system according to claim 10, wherein the universal connector is configured as a hollow helical wire-body sleeve.

17. The system according to claim 10, wherein the connector distal opening and the connector lumen accommodate reception therein of guidewire proximal portions having different proximal tip section configurations, for secure releasable retention.

18. The system according to claim 10, wherein a sleeve of flexible film of material covers at least the length of the universal connector for enhancing smooth passage thereover of implements.

19. The system according to claim 10, wherein the length of the connector body is coated with a coat of lubricant for enhancing smooth passage thereover of implements.

20. The system according to claim 10, wherein flexibility of the universal connector is controlled.
